(19) United States
(12) Patent Application Publication
(10) Pub. No.: US 2015/0202651 A1
(43) Pub. Date: Jul. 23, 2015

(54) Recombinant Spider Silk Protein Film and Method of Synthesizing

(71) Applicants: Randolph V. Lewis, Nibley, UT (US); Justin A. Jones, Nibley, UT (US); Chauncey L. Tucker, Logan, UT (US)

(72) Inventors: Randolph V. Lewis, Nibley, UT (US); Justin A. Jones, Nibley, UT (US); Chauncey L. Tucker, Logan, UT (US)

(73) Assignee: UTAH STATE UNIVERSITY, North Logan, UT (US)

(21) Appl. No.: 14/574,235

(22) Filed: Dec. 17, 2014

Related U.S. Application Data
(60) Provisional application No. 61/917,259, filed on Dec. 17, 2013.

(51) Int. Cl. B05D 3/06; A61K 8/64; A61K 35/646; B01D 29/00; A61L 27/22; G02B 1/10; C08J 5/18; B29C 35/08; A61Q 19/00

(52) U.S. Cl. CPC B05D 3/06; B29C 35/08; A61K 8/64; A61K 35/646; A61Q 19/00; A61L 27/227; G02B 1/10; C08J 5/18; B01D 29/0093; B29K 2093/00; B29L 2007/00

(57) ABSTRACT

Methods for forming useful films using recombinant spider silk protein are discussed. In one embodiment, the method involves dissolving silk protein in a sufficient quantity to form a film suspended in a solvent. The solution is then mixed with a compound selected from water, acids, imidazole crosslinking agents, antibiotics, nanoparticles, surfactants and combinations thereof. Solubilisation of the protein in the solution is effected by microwave exposure. Once solubilized the microwave treated solution is poured onto a suitable substrate and dried in order to result in the final film, the protein may be plant, mammal or bacterially derived. The film has widespread use and can be combined with any one of a number of substrates, examples of which include: a cosmetic composition; a pharmaceutical or medical composition, drug delivery system, artificial cell, contact lens coating, sustained-release drug delivery system, artificial skin graft; food composition; automotive part; aeronautic component; computer or data storage device, building material, textile, filter material, membrane material, nanomaterial, electronic component and combinations thereof.

### RELATED APPLICATIONS

This application claims priority to U.S. Provisional Patent Application No. 61/917,259, filed Dec. 17, 2013, the entirety of which is herein incorporated by reference. This application is related to U.S. patent application Ser. No. 14/459,244, filed Aug. 13, 2014, the entirety of which is herein incorporated by reference.

### FIELD OF THE INVENTION

The present invention is directed to a method of forming recombinant spider silk protein films and more particularly, the present invention is directed to methodology for synthesizing the film and uses of the film in various industries.

### BACKGROUND OF THE INVENTION

The use of recombinant spider silk proteins is well recognized in the art and has been set forth in U.S. Pat. No. 7,056,023, issued Jun. 6, 2006, to Islam et al., the contents of which is incorporated herein by reference. Generally, the disclosure explains methods and apparatuses for spinning silk protein fibers from recombinant proteins. The methods are primarily useful for spinning fibers of spider silk or silkworm silk proteins from recombinant mammalian cells and may be used to spin such fibers for use in the manufacture of industrial and commercial products.

Further examples of advancements in the art include that which is taught in U.S. Pat. No. 7,754,851, issued Jul. 13, 2010. In this reference, Scheibel et al. explain spider silk proteins, nucleic acids, coding for these recombinant spider silk proteins, as well as hosts suitable for expressing those nucleic acids. Further, there are discussions centred on a method of aggregation of spider silk proteins and the use of the proteins in the field of biotechnology and/or medicine and other industrial fields, particularly in the manufacture of automotive parts, in the aircraft construction, in the processing of textiles and leather, as well as in the manufacture and processing of paper. In United States Patent Application Publication US 2009/0263430, published Oct. 22, 2009, Scheibel et al., discuss a method of forming multilayer silk protein films and a multilayer film obtained therefrom. Various materials, products and compositions containing the multilayer film are also taught as well as the use of the film in several applications.

Other examples of the progress in this area of technology include developments evinced in U.S. Pat. Nos. 7,521,228; 5,989,894; 7,521,228; 5,989,894; 5,733,771; 5,756,677; 5,733,771; 5,756,677; 5,994,099 and 7,723,109 inter alia.

### SUMMARY OF THE INVENTION

One object of one embodiment of the present invention is to provide an improved recombinant spider silk protein film.

Another object of one embodiment of the present invention is to provide a new protocol for forming a recombinant spider silk protein film.

A still further object of one embodiment of the present invention ids to provide a method of forming silk protein film comprising the steps of:
a) providing a solution containing between 1 and 15% by weight of silk proteins dissolved or suspended in a suitable solvent;
b) mixing said solution with a compound selected from the group comprising water, acids, imidazole, crosslinking agents, antibiotics, nanoparticles, surfactants and combinations thereof;
c) exposing the solution to microwaves for a period sufficient to solubilize the protein;
d) pouring the microwave treated solution onto a substrate; and
e) drying said film.

Another object of one embodiment of the present invention is to provide a method of forming silk protein film comprising the steps of:
a) providing a solution containing between 1 and 15% by weight of silk proteins dissolved or suspended in a suitable solvent;
b) mixing said solution with a compound selected from the group comprising hexafluoroisopropanol (HFIP), acids, imidazole, crosslinking agents, antibiotics, nanoparticles, surfactants and combinations thereof;
c) mixing said solution for a period sufficient to solubilize the protein;
d) pouring the mixed solution onto a substrate; and
e) drying said film.

A still further object of one embodiment of the present invention is to provide a method for modifying mechanical property of a recombinant spider silk protein film, comprising:
a) providing a film made in accordance with claim 1:
b) providing an alcohol and water mixture;
c) conditioning said film in a conditioning step including stretching said film when said film is either within said mixture or out of said mixture; and
d) maintaining said film, when conditioned with said mixture, in said mixture for a period between 30 seconds and 10 minutes whereby a mechanical property of said film is modified relative to an unconditioned film.

A further object of one embodiment of the present invention is to provide a method for modifying mechanical property of a recombinant spider silk protein film, comprising:
a) providing a film made in accordance with claim 1;
b) providing an alcohol and water mixture;
c) conditioning said film in a conditioning step including stretching said film when said film is either within said mixture or out of said mixture; and
d) maintaining said film, when conditioned with said mixture, in said mixture for a period between 30 seconds and 10 minutes whereby a mechanical property of said film is modified relative to an unconditioned film.

Another object of the present invention is to provide a method for modifying mechanical property of a recombinant spider silk protein film, comprising:
a) providing a film made in accordance with claim 17;
b) providing an alcohol and water mixture;
c) conditioning said film in a conditioning step including stretching said film when said film is either within said mixture or out of said mixture; and
d) maintaining said film, when conditioned with said mixture, in said mixture for a period between 30 seconds and 10 minutes whereby a mechanical property of said film is modified relative to an unconditioned film.

These objects are solved by the subject-matter of the independent claims. Preferred embodiments are set forth in the dependent claims.

Having thus generally described the invention, reference will now be made to the accompanying drawings, illustrating preferred embodiments.

### BRIEF DESCRIPTION OF THE DRAWINGS

FIG. 1A is a view of a fiber spun where the dope had not been sonicated;

FIG. 1B is a view of a fiber spun where the dope had been sonicated;

FIG. 2 is a schematic illustration of the C-card used in the testing of the films;

FIG. 3 is a schematic illustration of the stretching device used for stretching the films;

FIG. 4 is an X-ray diffraction pattern for the film from Example II;

FIG. 5 is an illustration of the films from Example VI as positioned on a lawn of E. coli on LB agar;

FIG. 6 is an X-ray diffraction pattern for the film from Example VII;

FIG. 7 is an X-ray diffraction pattern for the film from Example XI;

FIG. 8 is an X-ray diffraction pattern for the film from Example XII;

FIG. 9 is an X-ray diffraction pattern for the film from Example XIII; and

FIG. 10 is an X-ray diffraction pattern for the film from Example XIV

Similar numerals employed in the drawings denote similar elements.

Unless otherwise defined, all technical and scientific terms used herein have the same meaning as commonly understood by one of ordinary skill in the art to which this invention pertains. All publications, patent applications, patents, and other references mentioned herein are incorporated by reference in their entirety. In case of conflict, the present specification, including definitions, will control. In addition, the materials, methods, and examples are illustrative only and not intended to be limiting.

### DETAILED DESCRIPTION OF THE PREFERRED EMBODIMENTS

The importance of spider silk for industrial applications cannot be overstated. It has ubiquitous utility in the processing of paper, cosmetics, food, electronic devices, drug delivery and in the automotive industry particularly for airbags and tires. Airbags, as is well known, are designed to push the passenger back into the seat without absorbing the full impact of the force. Employing spider silk in airbags, would provide more flexibility and absorb more energy. This could make airbags a more effective lifesaver.

In respect of tires, currently, tires have Kevlar cords on the inside which makes the tires strong and reliable. The problem with Kevlar it that it has a tendency to blow up, that is the reason why tires explode. Fibers made with spider silk in the tires would allow the tire to absorb more impact, making explosions unlikely.

Particularly convenient is the fact that the film can be combined with a innumerable examples of substrates such as a cosmetic composition, a pharmaceutical or medical composition, drug delivery system, artificial cell, contact lens coating, sustained-release drug delivery system, artificial skin graft; food composition; automotive part; aeronautic component; computer or data storage device, building material, textile, filter material, membrane material, nanomaterial, electronic component and combinations thereof.

With the degree of activity in this area of technology and despite the voluminous amount of prior art that has been created, there still exists a need for expedient protocols for recombinant spider silk protein film synthesis for use in a wide variety of industries as well as a film synthesized in accordance with the protocols having superior mechanical properties. This would lead to use of the synthesized film material into the automotive, pharmacological, medical, manufacturing, food, clothing, electronics, inter alia.

Advantageously, the present invention in its many facets now presents an elegant synthesis protocol and product to address the void in this technology area.

### Protein Preparation

Prior to a presentation of the synthesis of films using the spider silk protein, one possible route for the formulation of the protein is discussed below for general background.

Recombinant spider silk proteins, rSSPs, are conventionally dissolved in a very harsh organic solvent, 1,1,1,3,3,3-hexafluoroisopropanol (HFIP), to create “dopes” that can be used to create fibers, films, gels and foams. HFIP has been widely used and accepted as it is the only solvent that: 1) dissolves rSSPs at high concentrations (30% w/v) providing uniformity between various groups testing data; 2) is sufficiently volatile and miscible to be removed rapidly from the forming fiber; and 3) leaves little to no residue behind that could interfere with fiber formation. In addition, rSSPs generally are insoluble in aqueous solutions after purification, necessitating an organic solvent that meets the criteria outlined in points 1 through 3. However, there are significant problems with solvating rSSPs in HFIP or other organic solvents.

Dissolving rSSP in HFIP and then using pressure to extrude the dope into a coagulation bath does not allow the appropriate structures to form (notably fl-sheets) to an extent that the fibers or films have to be post-spin processed multiple times to achieve protein structures that result in appreciable mechanical properties. For reference purposes, Lazaris et al. discuss Spider Sik Fibers Spun from Soluble Recombinant Sik Produced in Mammalian Cells, Science 295, 472-476 (2002) (herein after “Lazaris”); and Teule et al., Modifications of spider silk sequences in an attempt to control the mechanical properties of the synthetic fibers, J. Mater Sci, 42, 8974-8985 (2007) (herein after “Teule”).

Such fiber processing methodologies include extruding the fiber into a coagulation bath that may include pure isopropanol or a mixture of isopropanol:water. The fiber may then be stretched (1.5 to 6 times) in a second bath generally containing a mixture of isopropanol and water. A third bath may also be employed that contains pure water or a majority of water, and a second stretch applied in that bath (Lazaris). Water is the recurrent theme in these baths and it is the water that converts the helical structures present due to HFIP into strength providing fl-sheets (“Teule”).

The cost of purchase and subsequent disposal of HFIP may be restrictive or prohibitive in an industrial setting of mass production. The cost to purchase HFIP is approximately $1,000/100 mls of HFIP and 100 ml of HFIP would likely be capable of solvating 20-30 g of rSSP (20-30% w/v). Water is inexpensive even in its purest form. Referencing the MSDS published on Sigma Aldrich's web-site, disposal of HFIP requires; “Dissolve or mix the material with a combustible solvent and burn in a chemical incinerator equipped with an afterburner and scrubber,” a process that inherently has costs associated with it. Excess water can be evaporated or recycled and reused. Worker safety when utilizing such harsh, volatile solvents is also a consideration.

Further referring to the MSDS; “Material is extremely destructive to tissue of the mucous membranes and upper respiratory tract, eyes, and skin. Cough, Shortness of breath, Headache, Nausea” (SIC). Water has no such requirements. Finally, the process of producing rSSP products could not be considered “green” using HFIP. rSSPs are largely insoluble in water. There are a few notable exceptions: Teule describes a series of proteins (Y1S8 and A2S8) that were produced in bacteria and purified via Ni++ chromatography. Short fibers were pulled straight from the eluted, pure rSSP fraction. Lazaris describes ADF-3 (Araneus diadematus MaSp1) produced in mammalian cell culture. Water soluble ADF-3 was concentrated in the presence of glycine and extruded into a coagulation bath. A final example is a series of recombinant aciniform-like synthetic proteins that were able to be spun from an aqueous solution very similar to Teule 2007 (Xu 2012). Reference can be made to Xu et al., Recombinant Minimalist Spider Wrapping Silk Proteins Capable of Native-Like Fiber Formation. PloS-One 7(11): e50227. Doi: 10.1371/journal.pone.0050227 (2012). However, outside of this small sub-set of rSSPs, water solubility is elusive. Noteworthy is that the majority of these proteins were much smaller than the natural proteins and thus are unlikely to make mechanically useful fibers.

United States Patent Application Publication No. 2011/0230911, published Sep. 22, 2011, utilizes a top down approach using genetic manipulations and expression system manipulations to try and create water soluble silk proteins. Unfortunately, such processes are costly both in time to create the manipulations/cell lines and also the proteins appear to be expressed in mammalian cell cultures. The culture conditions for such cell lines are not only personnel and time intensive, but also the ingredients and equipment required are substantially more expensive than the more traditional bacterial expression systems. In addition, such methods are limiting as there are not that many iterations of various spider silk repeats that can be expressed in this manner that will result in a water soluble protein having appreciable mechanical properties.

To address these and other challenges, this discussion sets forth new and novel methods for solubilizing rSSPs in aqueous solutions and then creating resulting spider silk compositions therefrom. The methods and compositions described herein in embodiments create aqueous dopes from rSSPs that are otherwise not soluble in water. The methods and compositions described herein may be applied to proteins expressed by any organism, reducing the cost of production and also possibly improving the mechanical properties of the fibers, films, gels and foams by the inclusion of water in the dope.

In certain embodiments, methods of preparing aqueous dopes of rSSP may include the following steps: mixing rSSP, water, and optional additives; optionally sonicating the mixture; microwaving the mixture; and optionally centrifuging the microwaved mixture.

### Aqueous Dopes

rSSP and water are combined to create a doping mixture of greater than about 2% w/v (e.g. 0.02 g SSpS:1 mL H₂O). In embodiments, the w/v does not typically exceed 50%. However, any percentage of less than 50% may be used. Suitable rSSPs include: MaSp1 (as described in U.S. Pat. Nos. 7,521,228 and 5,989,894), MaSp2 (as described in U.S. Pat. Nos. 7,521,228 and 5,989,894), MiSp1 (as described in U.S. Pat. Nos. 5,733,771 and 5,756,677), MiSp2 (as described in U.S. Pat. Nos. 5,733,771 and 5,756,677), Flagelliform (as described in U.S. Pat. No. 5,994,099), chimeric rSSPs (as described in U.S. Pat. No. 7,723,109), Pyriform, aciniform, tubuliform, aggregate gland silk proteins, and AdF-3 and AdF-4 from Araneus diadematus. Each of the above referenced patents is herein incorporated by reference in its entirety.

### Dope Additives

Various additives may be optionally added to the mixture. Suitable additives include compositions that contribute to the solubility of the rSSP in the solution. Some additives break or weaken disulfide bonds, thereby increasing the solubility of rSSPs. Other additives also serve to prevent hydrogel formation after the completion of the microwave step, as set forth below. If the solution forms a hydrogel quickly and the desired end product is not a gel, then additives capable of delaying or inhibiting such a formation may be desirable. In some embodiments, multiple additives may be added to achieve desired end products.

For example, to combat hydrogel formation, various additives may be added to the suspension of rSSP and water prior to microwaving the suspension. In some embodiments, acid, base, free amino acids, surfactants, or combinations thereof may be employed to combat hydrogel formation. For example, additions of acid (formic acid and acetic acid alone or together at 0.1% to 10% v/v), base (ammonium hydroxide at 0.1% to 10% v/v), free amino acids (L-Arginine and L-Glutamic Acid at 1 to 100 mM) as well as a variety of surfactants (Triton X-100 at 0.1% v/v) may be used. The additions of these various chemicals not only aid the solubility of rSSP when microwaved but in certain combinations also delay the solution from turning into a hydrogel long enough for the solution to be spun into a fiber.

By altering and adjusting the combinations of additives to the dopes, the mechanical properties of the spun fiber are significantly iM Pacted. For example, too much acid or base may result in fibers that are brittle with little to no extensibility; too little acid or base may result in dopes where the rSSP will not solubilize to the extent necessary for fiber spinning or turns to a hydrogel quickly.

Exemplary additives also include compositions capable of breaking or weakening disulfide bonds, such as β-mercaptoethanol or dithiothreitol may be added to reduce bonds and increase solubility. Suitable amounts of such additives may include from about 0.1 to about 5% (v/v). In embodiments where the rSSP does not contain cysteine, the use of such additives may be unnecessary. In some embodiments employing major ampulate silk proteins 1 and 2 (MaSp1 and MaSp2, respectfully), disulfide bonds (cysteine) are present in the C-terminus of the non-repetitive regions of MaSp1 and MaSp2. These proteins are described in U.S. Pat. Nos. 7,521,228 and 5,989,894, the entirety of both being herein incorporated by reference. In addition, the C-term is present in various goat-derived spider silk proteins M4, M5 and M55 proteins, which are described in U.S. Pat. No. 7,157,615, issued Jan. 2, 2007, the entirety of which is incorporated by reference in its entirety. In some embodiments, formic acid and/or acetic acid may be included in as little as 0.3% (v/v) but even lower amounts (0.1% v/v) are possible. Additionally, it is possible to solubilize rSSP without using any additives.

Exemplary additives are set forth in Table 1 (below), where dope formulations prepared according to the methods described herein and their resultant fibers/films mechanical properties are listed.

TABLE 1
Additives

| | | 3 | 4 | |
|---|---|---|---|---|
| 1 | 2 | Free Amino | Disulfide | 5 |
| Acid | Base | Acids | Reduction | Other |
| Acetic | Ammonium | Arginine | β- | Triton X-100 |
| Formic | Hydroxide | Glutamic | mercapto- | Glutaraldahyde |
| Trifluoro- | Sodium | Acid | ethanol | Calcium |
| acetic | Hydroxide | Histidine | Dithiothreitol | Potassium |
| acid | | Glycine | | Other |
| Other | | Imidazole | | Surfactants |
| Organic | | Other Free | | Other Ions |
| Acids | | Amino Acids | | |

To formulate an aqueous solution of rSSP, additives can be chosen from any of the five columns. For instance one or a combination of acids can be chosen from column one and combined with one or combinations of free amino acids from column three, as well as disulphide reducing compounds from column four and “Other” additives as required by the particular protein. Generally, it would not be useful to include both an acid from column one with a base from column two. However, a base from column two can be combined with additives from columns three and four. In some embodiments free amino acid analogues may also be used in place of or in addition to other free amino acids. For example, imidazole anaolgues such as benzimidazole, dihydroimidazole (imidazoline), pyrrole, axazole, thiazole, pyrazole, and triazoles may be used.

### Sonicaton

In some embodiments, the mixture containing water, rSSPs, and optional additives may be sonicated. The addition of sonication to the rSSP and water suspension may greatly increase the amount of solublized protein. Sonication may be performed with any suitable sonicator, such as a Misonix 3000 with microtip at 3.0 watts) either prior to microwaving, after microwaving and cooling, or both.

In embodiments, sonication may be employed to improve the amount of rSSP solubilized and, thus, reduce the amount of protein required to form an aqueous spin dope. Sonication also has the added benefit of producing a more homogenous solution. Sonication also improves and/or changes mechanical properties for rSSP composition products, particularly fiber mechanical properties.

For example, initial experiments required a 12.5% w/v MaSp1 analogue (125 mg MaSp1 into 1 ml of aqueous) in order to spin a fiber. Sonicating after microwaving reduced the concentration of MaSp1 to 5% w/v necessary to form fibers. Lower rSSP concentrations results in more fiber spun from a given amount of protein as well as finer fibers which has been demonstrated to increase the mechanical properties in other systems (electrospinning from HFIP based dope solutions (Teule).

### Microwaving

The mixture containing water, rSSPs, and optional additives may be microwaved prior to or after the optional sonication step. In embodiments, any microwave may be employed. In some embodiments, the mixture should be sealed prior to microwaving so as to avoid evaporation.

The mixture may be microwaved for any suitable amount of time to achieve the desired end product. The time depends on the power of the microwave and the amount of solution to be microwaved. In some embodiments, the solution may be stirred or agitated during microwaving so as to evenly expose the mixture to the microwaves. Appropriate times per unit being microwaved include, for example, from 10 to 90 seconds per 1 milliter of mixture. In some embodiments the 1 ml mixture may be set at from about 10% to 100% power for from about 5 second to 120 seconds.

After microwaving, the solution is allowed to cool and/or is taken to other processing steps, depending on the desired product. In some alternative embodiments, microwaving may be replaced with a reactor, such as the Series 4590 Micro Stirred Reactor by Parr Instrument Company or a larger commercial reactor. In such embodiments, the reactor is configured to control mixing, pressure, and temperature parameters. Suitable mixing speeds, temperatures, and pressures may be exerted in such embodiments.

### Centrifugation

In some embodiments, the microwaved mixture may be optionally centrifuged. After centrifugation, the resulting supernatant may be removed and then used for rSSP compositions and further processing.

### Gel Formation

Hydrogels may be generated from aqueous rSSP solutions by allowing the solubilized rSSP to cool. Additives to the dope such as acetic or formic acid can delay the formation of the hydrogel to allow the rSSP to be transferred to a mold prior to gelation. Theoretically, the variety of shapes that can be generated is limitless. The additives to the solution will change the mechanical properties of the resulting hydrogel. Hydrogel formation has been observed in solutions with as little as 3% w/v rSSP:water and all iterations greater.

The higher the percentage of rSSP, the more rapidly the solution gelates. Work in other systems, Bombyx mori silk, has proven the phenomenon that increasing the ratio of silk to water improves the mechanical characteristics of the resulting hydrogel. As well, altering the temperature, pH and including calcium ions changes the properties of the gels (Kim, U J et al., 2004, Biomacromolecules “Structure and Properties of Silk Hydrogels” Biomacromolecules 5, 786-792).

An example of a hydrogel application is illustrated in Chao et al., “Silk Hydrogel for Cartilage Tissue Engineering.” Journal of Biomedical Materials Research Part B: Applied Biomaterials, Vol 95B, Issue 1 pg 84-90, 2010.

Aerogels may be formed by freezing and then lyopholizing a solution or hydrogel of rSSP. Theoretically, the shapes for these aerogels is also limitless as their starting hydrogels could be allowed to form in a mold and then frozen and lyophilized.

### Film Formation

Films may be produced by pouring a dope solution onto a substrate and allowing the water and other additives to evaporate. If it is desirable to remove the film from the substrate, PDMS or Teflon allow the removal of the films. A representative dope solution comprises 50 mg/ml MaSP1 analogue, 1% formic acid, 1% acetic acid. Films may be applied as coatings or utilized after removal from a substrate. Film formation will be discussed in greater detail hereinafter.

### Foam Formation

Foam may be generated from aqueous based solvents by a variety of methods and dope conditions. One method reduced to practice is to formulate a dope solution similar/identical to that described for film generation. That solution is then placed into a vacuum chamber and a vacuum applied. The solution quickly expands and forms a foam upon curing in the chamber. Additives to the dopes such as surfactants will influence final cell size and further treatment of the foam (alcohol) are possible to also change the final properties of the foams. It is also possible that foams can be generated by chemical means, mainly peroxidase reactions, to produce CO₂ that creates bubbles in the dope and upon curing a foam remains. As discussed in United States Patent Publication No. 20110230911, published on Sep. 22, 2011, Scheibel. This method is also influenced by additives such as surfactants and post formation treatments (alcohol). A final method is an extrusion method whereby the dope solution is mechanically mixed with air, or other gas, to produce foam. This method is also subject to additives and post formation treatments to alter the final foam product.

### Fiber Formation

Fibers can be spun from aqueous solutions of rSSP by extrusion into a coagulation bath (alcohol) in a similar fashion as HFIP/aqueous based solutions of rSSP as described in United States Patent Application Publication No. 2005/0054830, published on Mar. 10, 2005. To summarize, the solubilized rSSP can be loaded into a syringe or other suitable extrusion instrument and then pushed through a fine bore needle into a bath comprised of isopropanol or other alcohol. As the rSSP drops through the alcohol, water is removed and a fiber is formed. That fiber can then be taken up or processed further by stretching in a second or even third bath comprised of alcohol(s), alcohol(s) and water or just water. Fibers have been formed from solutions with as little as 5% w/v solutions of rSSP:water. Similar 5% w/v solutions using HFIP as the solvent will not form fibers.

In some embodiments, it is not necessary for the solution to remain liquid to form fibers. Indeed, in some embodiments, fibers may be formed from a hydrogel. For example, when forming fibers from MaSp2 proteins, the process may be stopped, the syringe immediately removed for visualization, and a hydrogel may be observed. In contrast, forming fibers from a hydrogel with MaSp1 proteins results in deleterious effects.

It is important to note that each individual rSSP, due to its unique amino acid sequence, will have different requirements for aqueous solubility. The rSSP concentration, microwave time and power setting, amount of acid or base, and requirements for free amino acids or surfactants will be different. There does not appear to be one set of additives that achieves aqueous solubility and that also delays hydrogel formation for all rSSPs.

As an example, a 12.5% w/v solution of a MaSp1 and MaSp2 analogue can be prepared identically in terms of additives. The MaSp1 will become soluble in water easily and stay liquid for an extended period of time. The MaSp2, on the other hand, will form a hydrogel within minutes of removal from the microwave and requires more microwave time to solubilize.

All examples in this document are illustrative only and are not intended to limit the disclosure in any way.

### Process Example

### Dope Preparation

An aqueous recombinant spider silk protein (rSSP) dope solution was prepared by weighing out the rSSP such that a mass concentration of between 1 and 40% (w/v) of protein was achieved in 1 ml of water. For example, 50 mg of protein in 1 ml of water yielded a 5% w/v solution of protein to water. The suspension of rSSP and water was sealed inside a 3 ml glass Wheaton vial using a PTFE lined cap. The suspension and vial were then placed in a conventional 1500 watt microwave and microwaved at 50% power for 30 seconds. This solubilized the protein powder in the water.

Although this method may work to solubilize the rSSP, the solution quickly formed a hydrogel upon cooling and was generally not available thereafter to spin fibers by extrusion. If the goal of generating the aqueous dope is to form films, foams, hydrogels or aerogels, this method may be acceptable. Microwave time may vary depending on the volume of the dope, rSSP used, additives chosen, and whether sonication is utilized.

### Process Example

### Sonication

The following samples were prepared, one of which was not sonicated:
Dope not sonicated (12.5% M4, 1% Acetic Acid, 1% formic acid, 50 mM L-Arg, Microwaved 30 seconds at 50% power, centrifuged at 6000 rpm for 3 minutes, 1.5× stretch, 40× objective);
Dope sonicated (5% M4, 1% acetic acid, 1% formic acid, 50 mM L-Arg, microwaved
35 seconds at 50% power, sonicated at power level 1.5 (3 Watts) for 1.5 min., microwaved 30 seconds at 50% power, centrifuged 1 min. at 6000 rpm, 1.5× stretch, 40× objective.

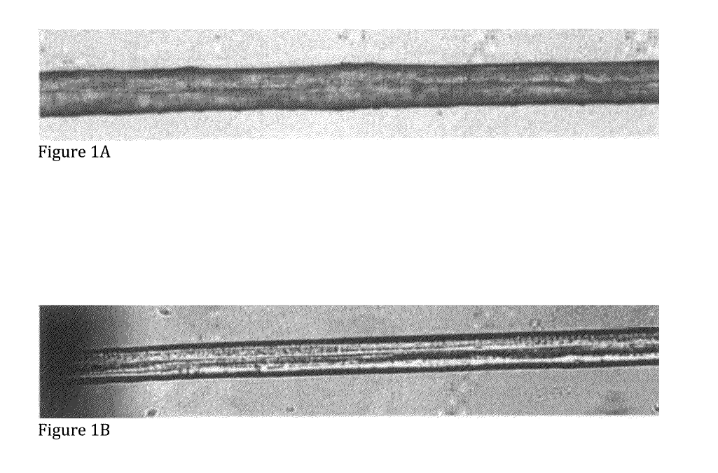

Fibers spun from dopes that are not sonicated (FIG. 1A), when analyzed microscopically, appear to have numerous lumps and discontinuities. The sonicated 5% w/v MaSP1 fibers (FIG. 1B) appear much more uniform. Sonication has the added benefit of requiring lower rSSP concentrations (5% coM Pared to greater than 8% without sonication) to spin fibers from lower concentrations are advantageous as less protein is used to spin similar lengths of fiber.

Thus, fiber defects when spun from aqueous dopes may be diminished by sonication of the dope.

The following examples set forth numerous rSSP sample tests and resulting data according the formulations and processing criteria set forth below:

### Example Set 1

25 mg of M5 (Nephila clavies MaSP2 analogue) was measured out using a fine balance into a 3 ml Wheaton glass vial with PTFE seal inside a plastic cap.
Included in the dope solution:
50 uL of 1M L-Arginine (L-Arginine is prepared in 18.2 MOhm water)
50 uL of glacial Acetic Acid (5% v/v)
900 uL of 18.2 MOhm water.

The PTFE sealed cap was placed on the 3 ml vial tightly. The solution and vial were placed into a conventional microwave (GE 1.6 kW) and microwaved for 30 seconds at 50% power. After microwaving, the solution was placed into a centrifuge (VWR Clinical 2000 set at 6,000 RPM) for 2 minutes to clarify. The supernatant is removed from any remaining pellet for spinning fibers or producing other materials such as films, gels or foams.

Fiber testing results (10 samples) for 1.5× post spin stretch in an 80:20 isopropanol:water bath.
Fiber testing results (9 samples) for 2.0× post spin stretch in an 80:20 isopropanol:water bath.
Diameter (μm) Energy to break (MJm⁻³) Max Stress (M Pa) Max Strain (mmmm⁻¹)

### Average 37.40 1.98 52.94 0.047

Standard deviation 2.27 1.43 0.03
Fiber testing results (9 samples) for 2.5× post spin stretch in an 80:20 isopropanol water bath.
Diameter (μm) Energy to break (MJm⁻³) Max Stress (M Pa) Max Strain (mmmm⁻¹)

### Average 37.25 0.75 60.05 0.02

Standard deviation 2.95 0.25 9.11 0.003
Diameter (μm) Energy to break (MJm⁻³) Max Stress (M Pa) Max Strain (mmmm⁻¹)

### Average 38.91 18.07 41.64 0.68

Standard deviation 5.15 14.64 17.17 0.54

Fiber testing results (10 samples) for 3.0× post spin stretch in an 80:20 isopropanol:water bath.

Fiber testing results (10 samples) for 3.5× post spin stretch in an 80:20 isopropanol:water bath.

### Example Set 2

125 mg of M5 (Nephila clavipes MaSP2 analogue) was measured out using a fine balance into a 3 ml Wheaton glass vial with PTFE seal inside a plastic cap.
Included in the dope solution:
50 μL of 1M L-Arginine (L-Arginine is prepared in 18.2 MOhm water)
100 μL of glacial Acetic Acid (10% v/v)
850 μL of 18.2 MOhm water.

The PTFE sealed cap was placed on the 3 ml vial tightly. The solution and vial were placed into a conventional microwave and microwaved for 30 seconds at 50% power. After microwaving, the solution was placed into a centrifuge (VWR Clinical 2000 set at 6,000 RPM) for 2 minutes to clarify.

The supernatant was removed from any remaining pellet for spinning fibers or producing other materials such as films, gels or foams.

Fiber testing results (9 samples) 1.5× post spin stretch in an 80:20 isopropanol:water bath.

Diameter (μm) Energy to break (MJm⁻³) Max Stress (M Pa) Max Strain (mmmm⁻¹)

### Average 26.71 40.25 84.54 0.57

Standard deviation 2.12 14.27 18.04 0.18
Diameter (μm) Energy to break (MJm⁻³) Max Stress (M Pa) Max Strain (mmmm⁻¹)

### Average 23.07 22.73 106.65 0.25

Standard deviation 2.64 8.76 22.91 0.09
Diameter (μm) Energy to break (MJm⁻³) Max Stress (M Pa) Max Strain (mmmm⁻¹)

### Average 34.17 4.52 72.88 0.07

Standard deviation 5.74 3.10 13.83 0.05

Fiber testing results (9 samples) 2.0× post spin stretch in an 80:20 isopropanol:water bath.

Fiber testing results (10 samples) 2.5× post spin stretch in an 80:20 isopropanol:water bath.

Fiber testing results (10 samples) 3.0× post spin stretch in an 80:20 isopropanol:water bath.

### Example Set 3

125 mg of M5 (Nephila clavipes MaSP2 analogue) was measured out using a fine balance into a 3 ml Wheaton glass vial with PTFE seal inside a plastic cap.
Included in the dope solution:
50 μL of 1M L-Arginine (L-Arginine is prepared in 18.2 MOhm water)
150 μL of glacial Acetic Acid (15% v/v)
800 μL of 18.2 MOhm water.
   Diameter (μm) Energy to break (MJm⁻³) Max Stress (M Pa) Max Strain (mmmm⁻¹)

### Average 31.02 3.92 74.05 0.08

Standard deviation 5.03 2.56 20.69 0.06
Diameter (μm) Energy to break (MJm⁻³) Max Stress (M Pa) Max Strain (mmmm⁻¹)

### Average 25.95 15.34 102.62 0.19

Standard deviation 1.08 13.71 17.87 0.18
Diameter (μm) Energy to break (MJm⁻³) Max Stress (M Pa) Max Strain (mmmm⁻¹)

### Average 25.71 41.89 87.67 0.55

Standard deviation 2.46 26.92 18.06 0.30

The PTFE sealed cap was placed on the 3 ml vial tightly. The solution and vial were placed into a conventional microwave and microwaved for 30 seconds at 50% power. After microwaving, the solution was placed into a centrifuge (VWR Clinical 2000 set at 6,000 RPM) for 2 minutes to clarify. The supernatant was removed from any remaining pellet for spinning fibers or producing other materials such as films, gels or foams.

Fiber testing results (10 samples) 1.5× post spin stretch in an 80:20 isopropanol:water bath.

Fiber testing results (10 samples) 2.0× post spin stretch in an 80:20 isopropanol:water bath.

Fiber testing results (10 samples) 2.5× post spin stretch in an 80:20 isopropanol:water bath.

### Example Set 4

125 mg of M5 (Nephila clavipes MaSP2 analogue) was measured out using a fine balance into a 3 ml Wheaton glass vial with PTFE seal inside a plastic cap.
Included in the dope solution:
50 μL of 1M L-Arginine (L-Arginine is prepared in 18.2 MOhm water)
200 μL of glacial acetic acid (20% v/v)
750 μL of 18.2 MOhm water.

The PTFE sealed cap was placed on the 3 mi vial tightly. The solution and vial were placed into a conventional microwave and microwaved for 30 seconds at 50% power.

Diameter (μm) Energy to break (MJm⁻³) Max Stress (M Pa) Max Strain (mmmm⁻¹)

### Average 39.20 3.77 69.92 0.07

Standard deviation 10.74 3.66 15.36 0.06
Diameter (μm) Energy to break MJm⁻³) Max Stress (M Pa) Max Strain (mmmm⁻¹)

### Average 46.93 20.47 53.81 0.37

Standard deviation 5.23 23.18 14.17 0.35
Diameter (μm) Energy to break (MJm⁻³) Max Stress (M Pa) Max Strain (mmmm⁻¹)

### Average 46.05 24.95 52.00 0.49

Standard deviation 6.42 25.25 16.49 0.43

After microwaving, the solution was placed into a centrifuge (VWR Clinical 2000 set at 6,000 RPM) for 2 minutes to clarify. The supernatant was removed from any remaining pellet for spinning fibers or producing other materials such as films, gels or foams.

Fiber testing results (10 samples) 1.5× post spin stretch in an 80:20 isopropanol:water bath.

Fiber testing results (9 samples) 2.0× post spin stretch in an 80:20 isopropanol:water bath.

Fiber testing results (10 samples) 3.5× post spin stretch in an 80:20 isopropanol:water bath.

### Example Set 5

125 mg of M4 (Nephila clavies MaSP1 analogue) was measured out using a fine balance into a 3 ml Wheaton glass vial with PTFE seal inside a plastic cap.
Included in the dope solution:
50 μL of 1M L-Arginine (L-Arginine is prepared in 18.2 MOhm water)
10 μL of glacial acetic acid (1% v/v)
10 μL of 88% Formic acid (1% v/v)
830 μL of 18.2 MOhm water.

The PTFE sealed cap was placed on the 3 ml vial tightly. The solution and vial were placed into a conventional microwave and microwaved for 30 seconds at 50% power. After microwaving, the solution was placed into a centrifuge for 5 minutes to clarify. The Diameter (μm) Energy to break (MJm⁻³) Max Stress (M Pa) Max Strain (mmmm⁻¹).

### Average 46.38 0.26 33.18 0.014

Standard deviation 10.11 0.11 7.64 0.003
Diameter (μm) Energy to break (MJm⁻³) Max Stress (M Pa) Max Strain (mmmm⁻¹)

### Average 44.65 1.09 63.71 0.02

Standard deviation 8.29 1.39 32.07 0.009
Diameter (μm) Energy to break (MJm⁻³) Max Stress (M Pa) Max Strain (mmmm⁻¹)

### Average 37.44 8.44 80.85 0.13

Standard deviation 2.04 11.70 8.09 0.16 supernatant was removed from any remaining pellet for spinning fibers or producing other materials such as films, gels or foams.

Fiber testing results (10 samples) 1.5× post spin stretch in an 80:20 isopropanol:water bath.

Fiber testing results (10 samples) 2.0× post spin stretch in an 80:20 isopropanol:water bath.

Fiber testing results (10 samples) 2.5× post spin stretch in an 80:20 isopropanol:water bath.

Fiber testing results (9 samples) 3.0× post spin stretch in an 80:20 isopropanol:water bath.

### Example Set 6

125 mg of M4 (Nephila clavipes MaSP1 analogue) was measured out using a fine balance into a 3 ml Wheaton glass vial with PTFE seal inside a plastic cap.
Included in the dope solution:
50 μL of 1M L-Arginine (L-Arginine is prepared in 18.2 MOhm water)
10 μL of glacial acetic acid (1% v/v)
   Diameter (μm) Energy to break (MJm⁻³) Max Stress (M Pa) Max Strain (mmmm⁻¹)

### Average 43.25 2.25 31.68 0.08

Standard deviation 16.23 1.25 7.83 0.04
Diameter (μm) Energy to break (MJm⁻³) Max Stress (M Pa) Max Strain (mmmm⁻¹)

### Average 30.02 5.02 61.68 0.09

Standard deviation 2.71 4.79 15.99 0.07
Diameter (μm) Energy to break (MJm⁻³) Max Stress (M Pa) Max Strain (mmmm⁻¹)

### Average 28.44 20.93 73.15 0.30

Standard deviation 3.40 18.50 30.78 0.16
Diameter (μm) Energy to break (MJm⁻³) Max Stress (M Pa) Max Strain (mmmm⁻¹)

### Average 27.57 3.85 33.38 0.14

Standard deviation 3.88 3.49 21.80 0.08
30 μL of 88% formic acid (3% v/v)
810 μL of 18.2 MOhm water.

The PTFE sealed cap was placed on the 3 ml vial tightly. The solution and vial were placed into a conventional microwave and microwaved for 30 seconds The solution and vial were allowed to cool and then, the solution was sonicated using a microtip on a Misonix sonicator for 1 minute at a power setting of 1.5. The PTFE sealed cap was placed on the 3 ml vial tightly. The solution and vial were placed into a conventional microwave and microwaved for 30 seconds at 50% power. After microwaving, the solution was placed into a centrifuge for 5 minutes to clarify. The supernatant was removed from any remaining pellet for spinning fibers or producing other materials such as films, gels or foams.

Fiber testing results (8 samples) 1.5× post spin stretch in an 80:20 isopropanol:water bath.

Fiber testing results (9 samples) 2.0× post spin stretch in an 80:20 isopropanol:water bath.

Fiber testing results (10 samples) 2.5× post spin stretch in an 80:20 isopropanol:water bath.

Fiber testing results (10 samples) 3.0× post spin stretch in an 80:20 isopropanol:water bath.

Diameter (μm) Energy to break (MJm⁻³) Max Stress (M Pa) Max Strain (mmmm⁻¹)

### Average 37.05 0.44 58.36 0.01

Standard deviation 3.32 0.14 13.03 0.002
Diameter (μm) Energy to break (MJm⁻³) Max Stress (M Pa) Max Strain (mmmm⁻¹)

### Average 49.03 0.51 33.61 0.02

Standard deviation 2.45 0.16 3.24 0.006
Diameter (μm) Energy to break (MJm⁻³) Max Stress (M Pa) Max Strain (mmmm⁻¹)

### Average 39.26 2.43 65.19 0.05

Standard deviation 10.08 1.96 35.24 0.04
Diameter (μm) Energy to break (MJm⁻³) Max Stress (M Pa) Max Strain (mmmm⁻¹)

### Average 26.42 1.12 98.28 0.02

Standard deviation 2.27 0.19 13.89 0.002

### Example Set 7

50 mg (5% w/v) of M4 (Nephila clavipes MaSP1 analogue) was measured out using a fine balance into a 3 m Wheaton glass vial with PTFE seal inside a plastic cap.
Included in the dope solution:
50 μL of 1M L-Arginine (L-Arginine is prepared in 18.2 MOhm water)
10 μL of glacial acetic acid (1% v/v)
940 μL of 18.2 MOhm water.

The PTFE sealed cap was placed on the 3 ml vial tightly. The solution and vial were placed into a conventional microwave and microwaved for 30 seconds at 50% power. After microwaving and cooling for 5 minutes, the solution was sonicated for 1 minute at 3.0 watts. After microwaving, the solution was placed into a centrifuge for 2 minutes to clarify. The supernatant was removed from any remaining pellet for spinning fibers or producing other materials such as films, gels or foams.

Fiber testing results 1.5× post spin stretch in an 80:20 isopropanol:water bath.

Diameter (μm) Energy to break (MJm⁻³) Max Stress (M Pa) Max Strain (mmmm⁻¹)

### Average 26.35 0.74 59.87 0.02

Standard deviation 0.35 0.39 8.30 0.007

Fiber testing results 3.0× post spin stretch in an 80:20 isopropanol:water bath.

Diameter (μm) Energy to break (MJm⁻³) Max Stress (M Pa) Max Strain (mmmm⁻¹)

### Average 17.30 16.31 112.10 0.16

Standard deviation 1.15 12.92 16.81 0.12

### Example Set 8

80 mg (8% w/v) of M4 (Nephila clavies MaSP1 analogue) in addition to
20 mg (2% w/v) of M5 (Nephila clavipes MaSP2 analogue) was measured out using a fine balance into a 3 ml Wheaton glass vial with PTFE seal inside a plastic cap.
Included in the dope solution:
50 μL of 1M L-Arginine (L-Arginine is prepared in 18.2 MOhm water)
50 μL of glacial acetic acid (5% v/v)
940 μL of 18.2 MOhm water.

The PTFE sealed cap was placed on the 3 ml vial tightly. The solution and vial were placed into a conventional microwave and microwaved for 35 seconds at 50% power. After microwaving, the solution was placed into a centrifuge for 3 minutes to clarify. The supernatant was removed from any remaining pellet for spinning fibers or producing other materials such as films, gels or foams.

Fiber testing results 2.0× post spin stretch in an 80:20 isopropanol:water bath. Diameter (μm)

Energy to break (MJm⁻³) Max Stress (M Pa) Max Strain (mmmm⁻¹)

### Average 31.62 1.70 68.16 0.04

Standard deviation 5.59 0.42 14.59 0.003

Fiber testing results 2.5× post spin stretch in an 80:20 isopropanol:water bath.

Diameter (μm) Energy to break (MJm⁻³) Max Stress (M Pa) Max Strain (mmmm⁻¹)

### Average 29.05 10.63 80.98 0.16

Standard deviation 1.07 3.84 10.78 0.04

Fiber testing results 3.0× post spin stretch in an 80:20 isopropanol:water bath.

Diameter (μm) Energy to break (MJm⁻³) Max Stress (M Pa) Max Strain (mmmm⁻¹)

### Average 25.48 23.31 84.91 0.31

Standard deviation 1.85 17.46 6.96 0.23

Fiber testing results 3.5× post spin stretch in an 80:20 isopropanol:water bath.

Diameter (μm) Energy to break (MJm⁻³) Max Stress (M Pa) Max Strain (mmmm⁻¹)

### Average 27.60 14.92 79.61 0.21

Energy to break 3.88 10.83 37.40 0.13

The methods and compositions described herein may also be applied to other traditionally insoluble proteins. Exemplary proteins that may be used in these methods include naturally occurring and synthetic proteins associated with protein misfolding diseases such as prions (CWD, BSE, vBSE, Creutzfeldt-Jakob), Alzheimer's, and Parkinson's.

Additionally, synthetically produced G-protein couple receptors (GPCR) are difficult targets as they to suffer aqueous solubility issues. Approximately 40% of drugs produced today are targeted at GPCRs. The methods described herein may also be applied to such GPCR.

Having the above as a background for possible rSSP synthesis, the discussion will now focus on the films incorporating the rSSP.

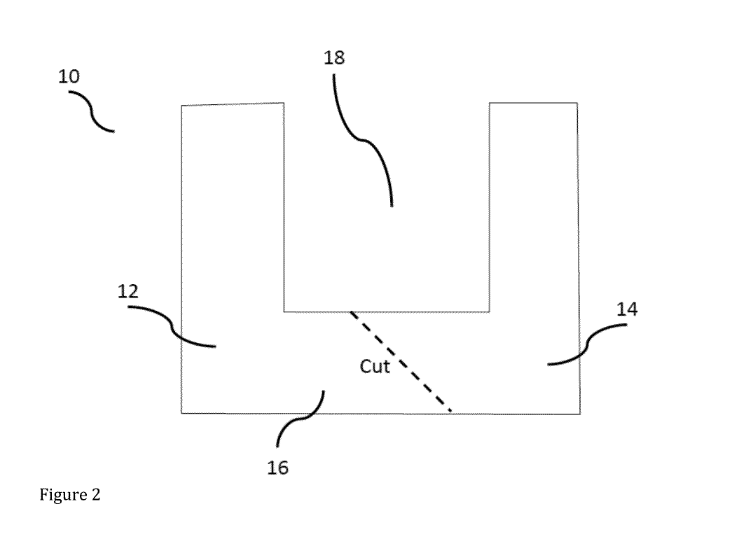

Referring initially to the drawings depicting the apparatus used in generating the films, FIG. 2 schematically illustrates the card for receiving the film to be tested. The C-card, referenced in greater detail herein after, is generally denoted by numeral 10 and includes a top section 12, bottom section 14 and connecting section 16 to thus define a “C” shape with an open area 18. In use, a sample of film (not shown) is cut to 3.5×13 mm and weighed to determine thickness. The film is then mounted on the C-card length wise using Loctite super glue (liquid). The prepared sample is then loaded on an MTS Synergie 100 using a 50N load cell, while clamping the top and bottom sides into the instrument and cutting the side so the only component tested is the film. The film is then tested running TestWorks 4 2001 at 5 mm/min and data collection at 30 Hz. A suitable material of which the card may be made is plastic, as an example.

Typical dimensions for the C-card 10 include an overall length of 19 mm, width of 9.5 mm with a depth of the open area being 6.5 mm and a height of 8 mm. These dimensions are, of course exemplary and specific for the testing devices used. In the event that alternative testing devices are employed, then there would be a commensurate change in the dimensions noted above.

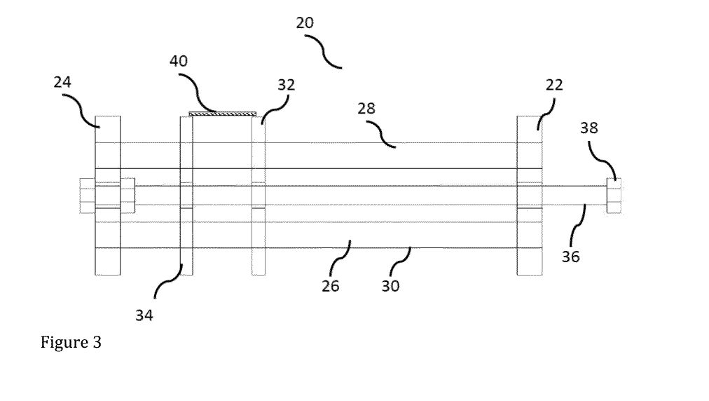

FIG. 3 illustrates an embodiment of the apparatus used for stretching the synthesized films. Numeral 20 globally references the device, which includes opposed support end members 22 and 24. A plurality of rod frame members 26, 28 and 30 extend between members 22 and 24 and are fixedly secured therein to define an open framework. A first 32 and second 34 receiving member are mounted on the frame rod members 26, 28 and 30 at least one of the receiving members 32 and/or 34 is movably mounted to the rod members 26, 28 and 30.

A threaded member 36 is mounted between end members 22 and 24 and extends through receiving members 32 and 34. The threaded member 36 may be actuated by manual or power assistance. In the embodiment depicted, a handle 38 is provided for manual actuation. Once rotated, at least one of the receiving members 22 and 24 moves either towards or away from the other depending on the direction of rotation of the handle 38 and thus threaded member 36.

Film samples 40 are shown mounted to the receiving members 22 and 24 and are mounted by suitable adhesive. The arrangement is effective to stretch a series of samples of film 40 consistently with equivalent force and simultaneously to ensure reproducibility in stretch results. Conveniently, owing to the portability of the arrangement 20, a sample loaded arrangement, such as that shown, may be immersed entirely in a solvent or the film exposed only (discussed herein after regarding film synthesis). Note that an automated version of this can be easily constructed from commonly available parts.

### Film Synthesis

Turning now to the synthesis of the films, each water based dope formed and used in the example where noted, contained between 1% and 15% protein to which water was added together with acids, crosslinking agents, antibiotics, nanoparticles and/or surfactants depending on the protein in order to maximize solubility, increase process ability, functionalize and/or customize mechanical properties. The dope was microwaved for a period of between 10 and 60 seconds in a sealed container optionally followed by multiple steps of sonication and further microwaving to liquefy the dope and solubilize all protein. This procedure can be applied to both goat and bacterially derived spider silk protein as well as spider silk protein from any other source.

Each water based dope was made in a sealed, microwavable vial capable of holding between 3 and 10 times the volume being made to prevent explosions. The solution contained between 1% and 15% or 10-150 mg/mL of recombinant spider silk protein to which was added water, acids, imidazole, crosslinking agents, antibiotics, nanoparticles and/or surfactants depending on the desired final product in order to maximize solubility, increase processability, functionalize and/or customize mechanical properties.

Each dope contained between 80% and 100% water and thus is referred to as a water-based dope. The vial containing the dope was sealed and microwaved for a period of between 10 and 60 seconds which optionally was followed by multiple steps of sonication and further microwaving to liquefy the dope and solubilize all protein. This procedure has been applied to both goat derived spider silk protein and bacterial-derived protein.

HFIP based dopes are also made in sealed vial but may be filled to the top. The vial with the HFIP based dope is set to mix overnight on a mini labroller. The solution contains 1% and 15% or 10-150 mg/mL protein to which is added HFIP, acids, crosslinking agents, antibiotics, nanoparticles and/or surfactants depending on the desired final product in order to maximize solubility, increase processability, functionalize and/or customize mechanical properties. Each dope contains between 80% and 100% HFIP and thus is called a HFIP-based dope.

### Film Synthesis Examples

### Example I

### Water-Based M4 (WBM4)

50 mg of goat generated M4 (MaSp1 in Nephila clavipes) powder was placed into a 3 mL Wheaton glass vial with PTFE seal inside a plastic lid. Included in the dope solution was 1 mL Nanopure water from a Thermo Fisher brand Barnstead. The plastic lid was then tightened onto the vial in order to prevent leaking. The vial and contents were then microwaved in a 1.6 kW GE household microwave oven for 1 minute. The dope solution was then poured, 200 μL a band, onto 4 bands of Sylgard 182 slicone elastomer Polydimethylsioxane (PDMS) 5:1 base: curing agent measuring 30×7 mm and allowed to dehydrate. After drying, the film was cut to 3.5×13 mm and weighed to determine thickness. The film was then mounted on the plastic C-card, discussed supra with respect to FIG. 1, length wise using Loctite super glue (liquid). The sample was then loaded on an MTS Synergie 100 using a 50N load cell, while clamping the top and bottom sides into the instrument and cutting the side so the only component tested was the film. The film was then tested running TestWorks 4 2001 at 5 mmmin⁻¹ and data collection at 30 Hz.
Results: Energy to break (MJm⁻³): 0.52; Stress (M Pa): 56.44; Strain (mmmm⁻¹): 0.02

### Example II

50 mg of M4 powder was placed into a 3 mL Wheaton glass vial with PTFE seal inside a plastic lid. Included in the dope solution was 999 mL Nanopure water from a Thermo Fisher brand Barnstead; 1 μL Formic acid, ACS, 88%+ from Alfa Aesar and 1 μL Glutaraldehyde (Added after centrifugation step). The plastic lid was then tightened onto vial to prevent leaking. The vial and contents were then microwaved in a 1.6 kW GE household microwave oven for 30 seconds The solution was then sonicated for 1.5 minutes at a power setting of 3 W using a Misonix sonictor 3000 with a microtip. The vial and contents were then microwaved in a 1.6 kW GE household microwave oven for 30 seconds. The solution was then sonicated for 1.5 minutes at a power setting of 3 W using a Misonix sonictor 3000 with a microtip. The vial and contents were then microwaved in a 1.6 kW GE household microwave oven for 30 seconds. The vial was then placed in a VWR 50 mL centrifuge tube with a 2 Kimwipes at the bottom for cushion. The centrifuge tube and contents were then placed in a VWR Clinical 200 centrifuge with a balance tube on the other side and centrifuged for 1 minute at 6000 RPM. Glutaraldehyde was then added to the vial and gently shaken to form a homogeneous mixture.

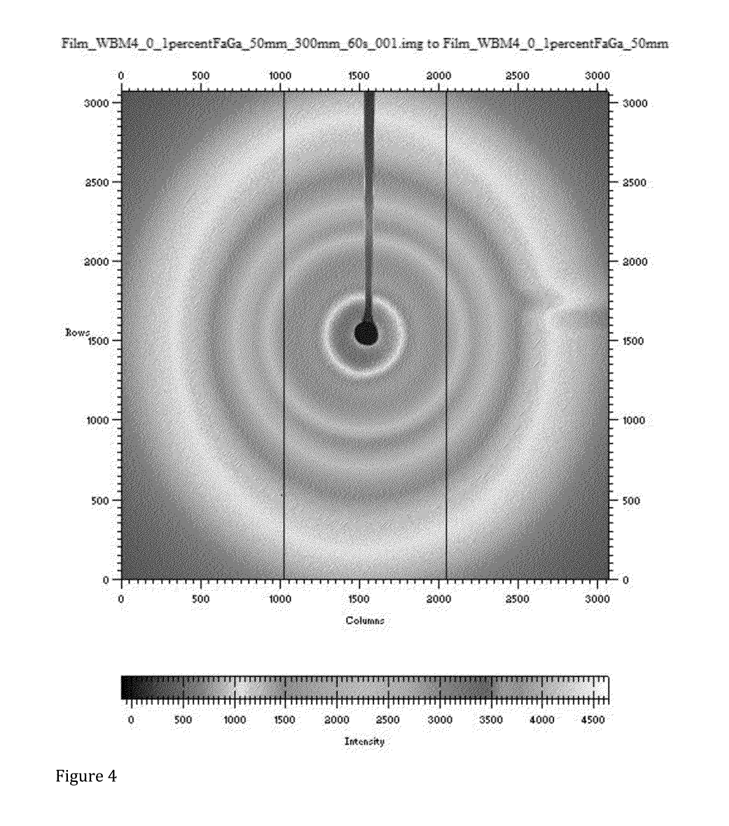

The dope solution was then poured with 200 μL per band onto 4 bands of Sylgard 182 silicone elastomer Polydimethylsiloxane (PDMS) 5:1 base: curing agent measuring 30×7 mm and allowed to dehydrate. After the drying stage, the film was prepared and tested as noted in the previous examples. The X-ray diffraction pattern is shown in FIG. 4. The pattern illustrates some beta sheet, but there is no orientation as noted by the uniform density around the ring.
Results: Energy to break: 0. MJm⁻³; Stress: 65.91 M Pa; Strain (mmmm⁻¹): 0.02

### Example III

50 mg of M4 powder was introduced into a 3 mL Wheaton glass vial with PTFE seal inside a plastic lid. Included in the dope solution was 999 mL Nanopure water from a Thermo Fisher brand Barnstead; 1 μL Formic acid, ACS, 88%+ from Alfa Aesar and 1 μL Glutaraldehyde (Added after centrifugation step). The plastic lid was then tightened onto vial to prevent leaking. The vial and contents were then microwaved as previous examples have delineated.

The solution was sonicated for 1.5 minutes at a power setting of 3 W using a Misonix sonicator 3000 with a microtip. The vial and contents were then microwaved in a 1.6 kW GE household microwave oven for 30 seconds. The vial was then placed in a VWR 50 mL centrifuge tube with a 2 Kimwipes at the bottom for cushion. The centrifuge tube and contents were then placed in a VWR Clinical 200 centrifuge with a balance tube on the other side and centrifuged for 1 minute at 6000 RPM. Glutaraldehyde was then added to the vial and gently shaken to form a homogeneous mixture.

The dope solution was then poured with 200 μL per band, onto 4 bands of Sylgard 182 silicone elastomer Polydimethylsiloxane (PDMS) 5:1 base: curing agent measuring 30×7 mm and allowed to dehydrate. After the drying stage, the film was cut in half, length wise and glued onto the stretching device referenced in FIG. 3 discussed supra using Loctite superglue (liquid). The device was then turned top side down, dipping the films into a solution of 50% Isopropanol and 50% water for 1 minute. The film was stretched from 8.5 to 29.75 mm (3.5×). After stretching, the films were dried with a Kimwipes. The stretching device was then turned top side down into warm (60° C.) water and left to soak for 1 minute. After soaking, the films were dried with a Kimwipes and cut off of the stretching device and 15 mm cut out of the middle of the film. The film was then mounted on the plastic C-card supra. The sample was then loaded on an MTS Synergi similar to the previous examples.
Results: Energy to break (MJm⁻³): 42.4; Stress (M Pa): 280.27; Strain (mmmm⁻¹): 0.21

### Example IV

50 mg of M4 powder was introduced into a 3 mL Wheaton glass vial with PTFE seal inside a plastic lid. Included in the dope solution is 999 mL Nanopure water from a Thermo Fisher brand Barnstead; 1 μL Formic acid, ACS, 88%+ from Alfa Aesar and 1 μL Glutaraldehyde (Added after centrifugation step). The plastic lid was then tightened onto vial to prevent leaking. The vial and contents were then microwaved in a 1.6 kW GE household microwave oven for 45 seconds. The vial was then placed in a VWR 50 mL centrifuge tube with a 2 Kimwipes at the bottom for cushion. The centrifuge tube and contents were then placed in a VWR Clinical 200 centrifuge with a balance tube on the other side and centrifuged for 1 minute at 6000 RPM. Glutaraldehyde was then added to the vial and gently shaken to form a homogeneous mixture.

The dope solution was then poured with 200 μL per band, onto 4 bands of Sylgard 182 silicone elastomer Polydimethylsiloxane (PDMS) 5:1 base: curing agent measuring 30×7 mm and allowed to dehydrate. After drying, the film was cut in half, length wise and glued onto the stretching device of FIG. 2. The device was then turned top side down, dipping the films into a solution of 50% Isopropanol and 50% water for 1 minute. The stretching device was then rotated top side up and the films stretched. This procedure was repeated to include separate stretches, namely (i) 8.5 to 21.25 mm (2.5×); (ii) 8.5 to 25.5 mm (3×) and (iii) 8.5 to 29.75 mm (3.5×). After stretching, the films were cut off of the stretching device and 15 mm removed from the middle of the film. The film was then mounted on the plastic C-card in a similar manner to that established in earlier referenced examples.

The sample was then loaded on an MTS Synergie as previously noted and tested the same way as that discussed from the previous examples.

### Results:

2.5× Stretch: Energy to break (MJm⁻³): 30.44; Stress (M Pa): 136.66; Strain (mmmm⁻¹): 0.25
3× Stretch: Energy to break (MJm⁻³): 10.47; Stress (M Pa): 91.53; Strain (mmmm⁻¹): 0.14
3.5× Stretch: Energy to break (MJm⁻³): 42.52; Stress (M Pa): 165.9; Strain (mmmm⁻¹): 0.30

### Example V

50 mg of M4 powder was introduced into a 3 mL Wheaton glass vial with PTFE seal inside a plastic lid. Included in the dope solution was 999 mL Nanopure water from a Thermo Fisher brand Barnstead; 1 μL Formic acid, ACS, 88%+ from Alfa Aesar; 1 μL Glutaraldehyde (Added after centrifugation step) and 100 μL gold nanoparticles (20 nm) from Ted Pella, Inc. (Added after centrifugation step). The plastic lid was then tightened onto vial to prevent leaking. The vial and contents were then microwaved in a 1.6 kW GE household microwave oven for 30 seconds. The vial was then placed in a VWR 50 mL centrifuge tube with a 2 Kimwipes at the bottom for cushioning purposes. The centrifuge tube and contents were then placed in a VWR Clinical 200 centrifuge with a balance tube on the other side and centrifuged for 1 minute at 6000 RPM. Gold nanoparticles were added to the solution. Glutaraldehyde was then added to the vial and gently shaken to form a homogeneous mixture.

The dope solution was then poured with 200 μL per band, onto 4 bands of Sylgard 182 silicone elastomer Polydimethylsiloxane (PDMS) 5:1 base: curing agent measuring 30×7 mm and allowed to dehydrate. After the drying stage, the film was cut in half, length wise and glued onto the stretching device in a similar manner to that disclosed in the previous examples. The device was then turned top side down, dipping the films into a solution of 50% Isopropanol and 50% water for 1 minute. The film was stretched from 8.5 to 29.75 mm (3.5×). After stretching, the stretching apparatus was turned right side up and the films dried with a Kimwipe. The films were then treated with steam in place for 5 minutes.

After steaming, the stretching apparatus was slackened from 29.75 to 28.5. The films were then dried with a Kimwipe and cut off the stretching device with 15 mm removed from the middle of the film. The film was then mounted on the plastic C-card as discussed previously.

The sample was then loaded on an MTS Synergie as noted previously.

Results: Energy to break (MJm⁻³): 7.51; Stress (M Pa): 116.74; Strain (mmmm⁻¹): 0.08

### Example VI

50 mg of goat produced M4 (MaSp1 in Nephila clavipes) powder was introduced into a 3 mL Wheaton glass vial with PTFE seal inside a plastic lid. Included in the dope solution was 999 mL Nanopure water from a Thermo Fisher brand Barnstead; 1 μL Formic acid, ACS, 88%+ from Alfa Aesar and 1 μL Kanamycin stock (15 mg/ml) (Added after centrifugation step). The plastic lid was then tightened onto vial to prevent leaking. The vial and contents were then microwaved in a 1.6 kW GE household microwave oven for 30 seconds.

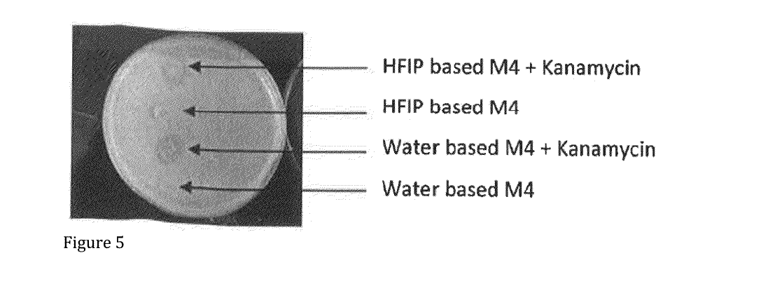

The dope solution was then poured with 200 μL a band onto 4 bands of Sylgard 182 silicone elastomer Polydimethylsiloxane (PDMS) 5:1 base: curing agent measuring 30×7 mm and allowed to dehydrate. After drying, 7 mm discs were punched out of the films. The disc was then put onto a lawn of E. coli grown on LB agar. The result is illustrated in FIG. 5.

### Example VII

### Water Based M5 (WBM5)

50 mg of goat produced M5 (MaSp2 in Nephila clavipes) powder was placed into a 3 mL Wheaton glass vial with PTFE seal inside a plastic lid. Included in the dope solution was 850 μL Nanopure water from a Thermo Fisher brand Barnstead; 50 μL Acetic acid and 100 μL 50M L-Arginine. The plastic lid was then tightened onto vial to prevent leaking. The vial and contents were then microwaved in a 1.6 kW GE household microwave oven for 15 seconds. The centrifuge tube and contents were then placed in a VWR Clinical 200 centrifuge with a balance tube on the other side and centrifuged for 1 minute at 4185 g.

The dope solution was then poured with 200 μL per band, onto 4 bands of Sylgard 182 silicone elastomer Polydimethylsiloxane (PDMS) 5:1 base: curing agent measuring 30×7 mm and allowed to dehydrate. After drying, the film is cut to 3.5×13 mm and weighed to determine thickness. The film was then mounted on the plastic C-card as noted previously.

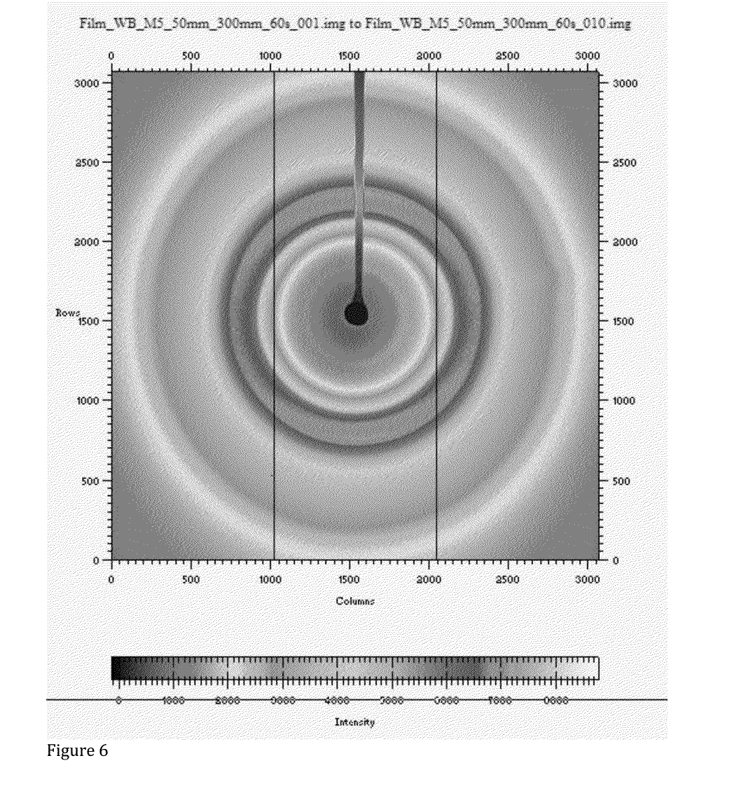

The sample was then loaded on an MTS Synergie following similar parameters as above. The X-ray diffraction data is shown in FIG. 6. The patterns illustrates that the film has increased beta sheet relative to X-Ray diffraction from Example II.
Results: Energy to break (MJm⁻³): 0.67; Stress (M Pa): 41.60; Strain (mmmm⁻¹): 0.03

### Example VIII

40 mg of goat produced M5 (MaSp2 in Nephila clavipes) powder was introduced into a 3 mL Wheaton glass vial with PTFE seal inside a plastic lid. Included in the dope solution was 800 μL Nanopure water from a Thermo Fisher brand Barnstead; 0.5 μL Formic acid; 200 μL 1M Imidazole and 0.5 μL Glutaraldehyde (Added after centrifugation step). The plastic lid was then tightened onto vial to prevent leaking. The vial and contents are then microwaved in a 1.6 kW GE household microwave oven for 30 seconds. The centrifuge tube and contents were then placed in a VWR Clinical 200 centrifuge with a balance tube on the other side and centrifuged for 1 minute at 4185 g.

The dope solution was then poured with 200 μL per band, onto 4 bands of Sylgard 182 silicone elastomer Polydimethylsiloxane (PDMS) 5:1 base: curing agent measuring 30×7 mm and allowed to dehydrate. After the drying stage, the film was cut to 3.5×13 mm and weighed to determine thickness. The film was then mounted on the plastic C-card and tested as noted previously.

### Example IX

### Water Based A4S8

50 mg of E. coli produced A4S8 (derived from MaSp2 and Flagelliform in Nephila clavipes) powder into a 3 mL Wheaton glass vial with PTFE seal inside a plastic lid. Included in the dope solution was 1 mL Nanopure water from a Thermo Fisher brand Barnstead and 1 μL Formic acid, ACS, 88%+ from Alfa Aesar. The plastic lid was then tightened onto vial to prevent leaking. The vial and contents were then microwaved in a 1.6 kW GE household microwave oven for 15 seconds.

The solution was then sonicated for 1.5 minutes at a power setting of 3 W using a Misonix sonictor 3000 with a microtip. The vial and contents were then microwaved in a 1.6 kW GE household microwave oven for 15 seconds. The solution was then sonicated for 1.5 minutes at a power setting of 3 W using a Misonix sonictor 3000 with a microtip. The vial and contents were then microwaved in a 1.6 kW GE household microwave oven for 20 seconds. The vial was then placed in a VWR 50 mL centrifuge tube with a 2 Kimwipes at the bottom for cushion. The centrifuge tube and contents were then placed in a VWR Clinical 200 centrifuge with a balance tube on the other side and centrifuged for 3 minutes at 4185 g.

The dope solution was then poured with 400 μL per band, onto 4 bands of Sylgard 182 silicone elastomer Polydimethylsioxane (PDMS) 5:1 base: curing agent measuring 30×7 mm and allowed to dehydrate. After the drying stage, the film was cut to 3.5×13 mm and weighed to determine thickness. The film was then mounted on the plastic C-card and tested as previously disclosed.
Results: Energy to break (MJm⁻³): 0.27; Stress (M Pa): 41.37; Strain (mmmm⁻¹): 0.01

### Example X

### HFIP Based M4

50 mg of goat produced M4 (MaSp1 in Nephila clavipes) powder was introduced into a 3 mL Wheaton glass vial with PTFE seal inside a plastic lid. Included in the dope solution is 1 mL HFIP. The plastic lid was then tightened onto vial to prevent leaking. The vial and contents were then mixed overnight on a mini labroller.

The dope solution was then poured, 200 μL a well, into 4 wells of Sylgard 182 silicone elastomer Polydimethylsiloxane (PDMS) mold using 5:1 base: curing agent measuring 30×7×0.3 mm and allowed to dehydrate. After the drying stage, the film was cut to 3.5×13 mm and weighed to determine thickness. The film was on the plastic C-card and tested as previously noted.
Results: Energy to break (MJm⁻³): 1.07; Stress (M Pa): 40.85; Strain (mmmm⁻¹): 0.05

### Example XI

50 mg of goat produced M4 (derived from MaSp1 in Nephila clavipes) powder was introduced into a 3 mL Wheaton glass vial with PTFE seal inside a plastic lid. Included in the dope solution was 800 μL HFIP and 200 μL Formic acid, ACS, 88%+ from Alfa Aesar. The plastic lid was then tightened onto vial to prevent leaking. The vial and contents were then mixed overnight on a mini labroller.

The dope solution was then poured with 200 μL per well, into 4 wells of Sylgard 182 silicone elastomer Polydimethylsiloxane (PDMS) mold using 5:1 base: curing agent measuring 30×7×0.3 mm and allowed to dehydrate. After the drying stage, the film was cut to 3.5×13 mm and weighed to determine thickness. The film was then mounted on the plastic C-card and tested as previously discussed.

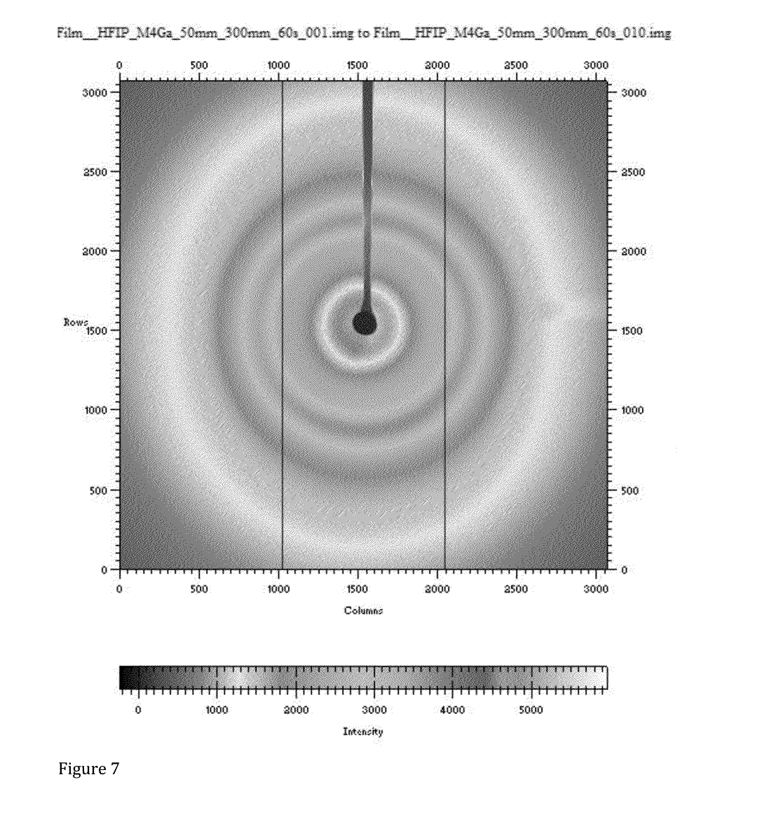

The X-ray diffraction pattern is shown in FIG. 7. The results of this X-Ray diffraction pattern are similar to those indicated with respect to Example II.
Results: Energy to break (MJm⁻³): 2.87; Stress (M Pa): 50.4; Strain (mmmm⁻¹): 0.08

### Example XII

50 mg of M4 (MaSp1 in Nephila clavipes) powder was introduced into a 3 mL Wheaton glass vial with PTFE seal inside a plastic lid. Included in the dope solution was 1 mL HFIP and 1 μL Glutaraldehyde (Added 10 minutes before pouring films). The plastic lid was then tightened onto vial to prevent leaking. The vial and contents were then mixed overnight on a mini labroller. Gluteraldehyde was added to the vial and gently shaken to homogenize the solution.

The dope solution was then poured with 200 μL per well, into 4 wells of Sylgard 182 silicone elastomer Polydimethylsiloxane (PDMS) mold using 5:1 base: curing agent measuring 30×7×0.3 mm and allowed to dehydrate. After the drying stage, the film was cut to 3.5×13 mm and weighed to determine thickness. The film was then mounted on the plastic C-card and tested as previously discussed.

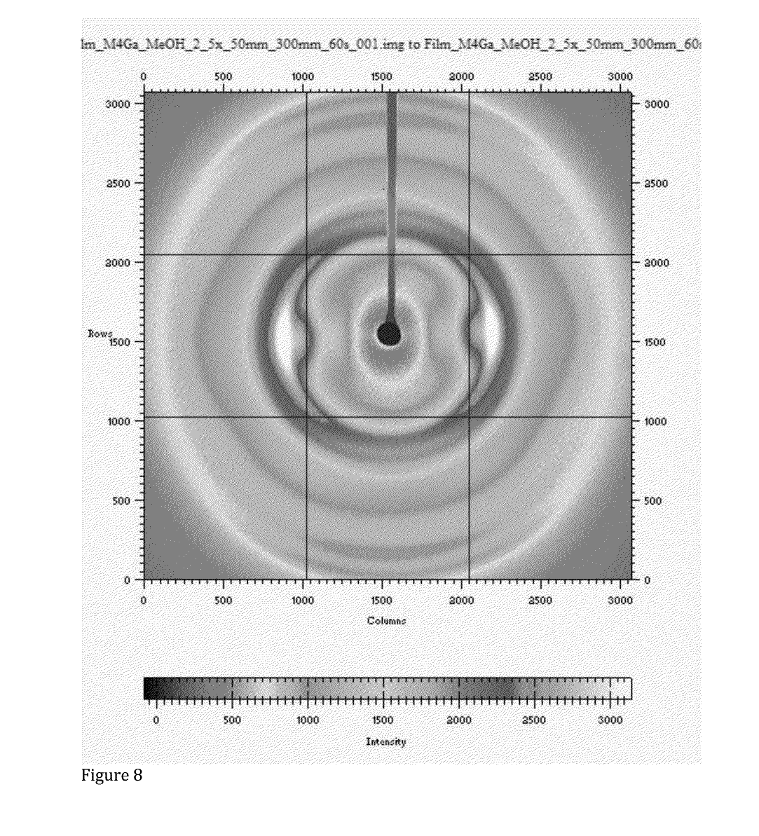

The X-ray diffraction pattern is shown in FIG. 8. This pattern shows both an increase beta sheet content and orientation as the density is higher than that in Examples II, VII and XI. The density is highly central to the equator which shows the sheets are oriented in the direction of the stretch of the film. Orientation factor 0.624 evinces this.
Results: Energy to break (MJm⁻³): 21.12; Stress (M Pa): 17.35; Strain (mmmm⁻¹): 1.638

### Example XIII

50 mg of goat produced M4 (MaSp1 in Nephila clavipes) powder was introduced into a 3 mL Wheaton glass vial with PTFE seal inside a plastic lid. Included in the dope solution was 1 mL HFIP and 1 μL Glutaraldehyde (Added 10 minutes before pouring films). The plastic lid was then tightened onto vial to prevent leaking. The vial and contents were then mixed overnight on a mini labroller. Gluteraldehyde was added to the vial and gently shaken to homogenize solution.

The dope solution was then poured with 200 μL per well, into 4 wells of Sylgard 182 silicone elastomer Polydimethylsiloxane (PDMS) 5:1 base: curing agent measuring 30×7×0.3 mm and allowed to dehydrate. After the drying stage, the excess was cut off the edges and the film cut in half, length wise and glued onto the stretching device as set forth previously.

The device was then turned top side down, dipping the films into a solution of 80% Methanol and 20% water for 30 seconds. The stretching device was then rotated top side up and the films stretched. This procedure was repeated to include separate stretches, namely (i) 8.5 to 17 mm (2×); (ii) 8.5 to 21.25 mm (2.5×) and (iii) 8.5 to 23.375 mm (2.75×). After stretching, the films were cut off of the stretching device and 15 mm removed from the middle of the film. The film was then mounted on the plastic C-card and tested as previously discussed.

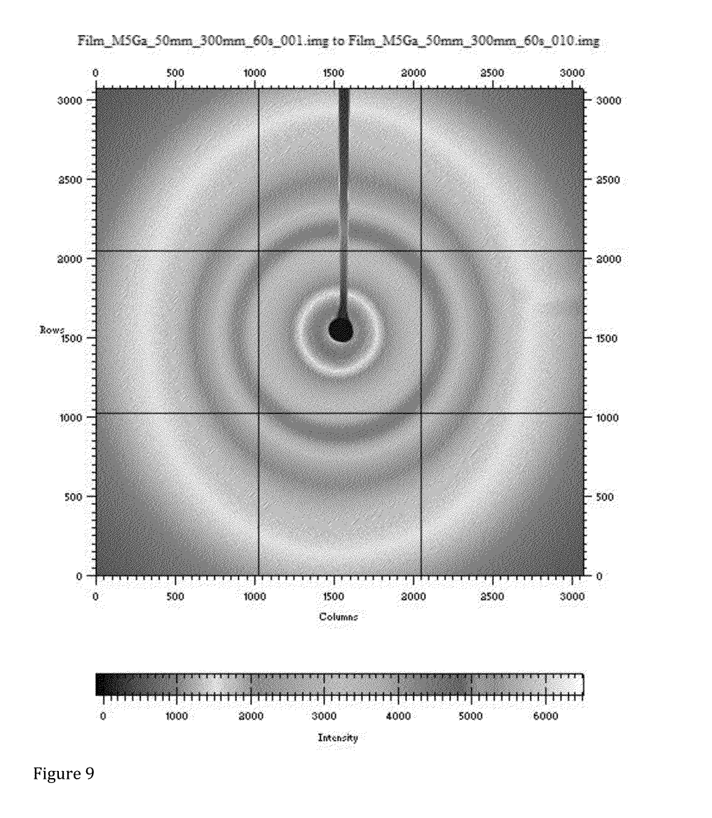

The X-ray diffraction pattern is shown in FIG. 9. The pattern shown is quite similar to that of Examples II, VII and XI.

### Results:

2× Stretch: Energy to break (MJm⁻³): 36.72; Stress (M Pa): 115.55; Strain (mmmm⁻¹): 0.37
2.5× Stretch: Energy to break (MJm⁻³): 39.39; Stress (M Pa): 189.18; Strain (mmmm⁻¹): 0.26
2.75× Stretch: Energy to break (MJm⁻³): 52.33; Stress (M Pa): 212.46; Strain (mmmm⁻¹): 0.32

### Example XIV

50 mg of M5 (MaSp2 in Nephila clavipes) powder was introduced into a 3 mL Wheaton glass vial with PTFE seal inside a plastic lid. Included in the dope solution was 1 mL HFIP and 1 μL Glutaraldehyde (Added 10 minutes before pouring films). The plastic lid was then tightened onto vial to prevent leaking. The vial and contents were then mixed overnight on a mini labroller. Gluteraldehyde was added to the vial and gently shaken to homogenize the solution.

The dope solution was then poured, 200 μL a well, into 4 wells of Sylgard 182 silicone elastomer Polydimethylsiloxane (PDMS) mold using 5:1 base: curing agent measuring 30×7×0.3 mm and allowed to dehydrate. After the drying stage, the film was cut to 3.5×13 mm and weighed to determine thickness. The film was then mounted on the plastic C-card and tested as previously discussed.

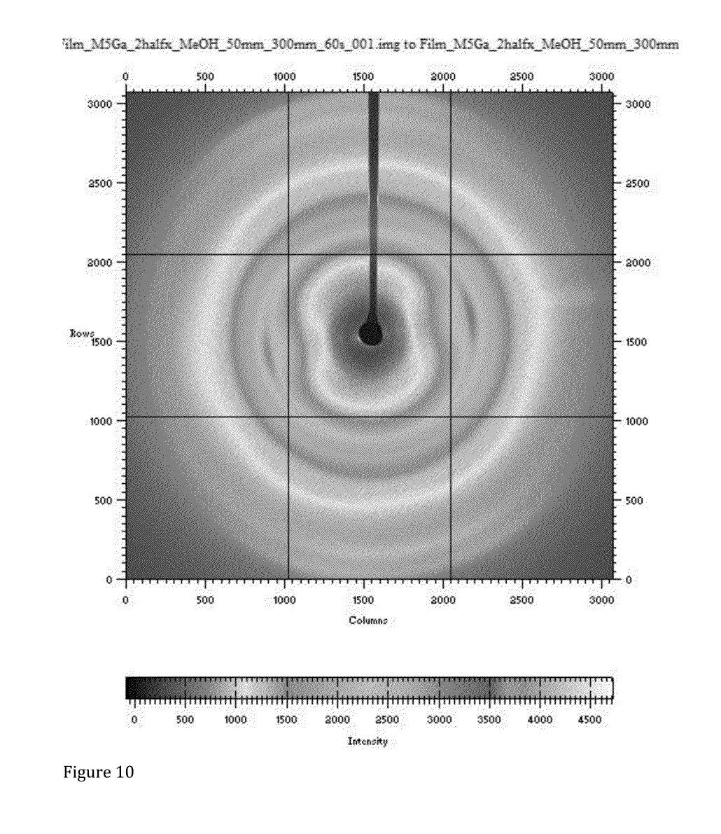

The X-ray diffraction pattern is shown in FIG. 10. A similar pattern is demonstrated as provided for in Example XII which shows orientation of the beta sheets parallel to the direction of the stretch. The orientation factor of 0.447 indicates this, but, it is less than in Example XII.
Results: Energy to break (MJm⁻³): 1.84; Stress (M Pa): 31.26; Strain (mmmm⁻¹): 0.08

### Example XV

50 mg of goat produced M5 (MaSp2 in Nephila clavipes) powder was introduced into a 3 mL Wheaton glass vial with PTFE seal inside a plastic lid. Included in the dope solution was 1 mL HFIP and 1 μL Glutaraldehyde (Added 10 minutes before pouring films). The plastic lid was then tightened onto vial to prevent leaking. The vial and contents are then mixed overnight on a mini labroller. Gluteraldehyde was added to the vial and gently shaken to homogenize the solution.

The dope solution was then poured with 200 μL per well, into 4 wells of Sylgard 182 silicone elastomer Polydimethylsiloxane (PDMS) 5:1 base: curing agent measuring 30×7×0.3 mm and allowed to dehydrate. After the drying stage, the excess was removed from the edges and the film cut in half, length wise and glued onto the stretching device of FIG. 2.

The device was then turned top side down, dipping the films into a solution of 80% Methanol and 20% water for 30 seconds. The stretching device was then rotated top side up and the films stretched. This procedure was repeated to include separate stretches, namely (i) 8.5 to 21.25 mm (2.5×) and (ii) 8.5 to 23.375 mm (2.75×). After stretching, the films were cut off of the stretching device and 15 mm removed from the middle of the film. The film was then mounted on the plastic C-card and tested as previously discussed.

### Results:

2.5× Stretch: Energy to break (MJm⁻³): 33.36; Stress (M Pa): 132.17; Strain (mmmm⁻¹): 0.33
2.75× Stretch: Energy to break (MJm⁻³): 16; Stress (M Pa): 117.87; Strain (mmmm⁻¹): 0.17

In conclusion, it can be seen that the protocol results in very significant increases in desirable mechanical properties in the film product completely capable of full integration into the vast industries discussed herein. As is further evident from the data presented herein, stretching of the films results in increased beta sheet with orientation in direction of the stretch. This attribute explains the substantial increase in the strength of the films.

Although the embodiments of the invention have been described above, it is limited thereto and it will be apparent to those skilled in the art that numerous modifications from part of the present invention insofar as they do not depart from the spirit, nature and scope of the described invention.

We claim:

1. A method of forming silk protein film, the method comprising:
providing a solution containing silk proteins in a quantity sufficient to form a firm dissolved or suspended in a suitable solvent;
mixing said solution with a compound selected from the group comprising water, acids, imidazole or its analogs, crosslinking agents, antibiotics, nanoparticles, surfactants, and combinations thereof;
exposing the solution to microwaves for a period sufficient to solubilize the protein;
pouring the microwave treated solution onto a substrate; and
drying said film.

2. The method as set forth in claim 1, wherein said solution contains between 1% and 60% by weight silk protein.

3. The method of claim 1, wherein said solution is a dope and contains at least 80% water by weight.

4. The method of claim 1, wherein said substrate comprises a polydimethysiloxane substrate.

5. The method of claim 1, further including the step of sonicating said mixture subsequent to microwave treatment.

6. The method of claim 1, further including the step of centifuging said mixture prior to pouring.

7. The method of claim 6, further including the step of homogenizing said mixture prior to pouring.

8. The method of claim 6, wherein homogenization is effected by the addition of a crosslinking compound to said mixture.

9. The method of claim 1, wherein drying comprises refrigeration drying or half sash fume hood drying.

10. The method of claim 1, wherein said spider silk proteins comprise a recombinant spider silk protein selected from the group consisting of MaSp 2, MaSp1, MaSp1 analogues, MaSp2 analogues, Flag and Flag analogues, MiSp and MiSp analogues, Piri and Piri analogs, and Acin and Acin analogs.

11. The method of claim 1, wherein said solution further includes additives for suppressing gel formation in the solution, said additives being selected from the group consisting of formic acid, acetic acid, ammonium hydroxide, L-arginine, L-glutamic acid, beta mercaptoethanol, dithiothreitol and combinations thereof.

12. A film made in accordance with the method of claim 1.

13. A method for modifying a mechanical property of a recombinant spider silk protein film, the method comprising:
providing a film made in accordance with claim 1;
providing an alcohol and water mixture;
conditioning said film in a conditioning step including stretching said film when said film is either within said mixture or out of said mixture; and
maintaining said film, when conditioned with said mixture, in said mixture for a period between 30 seconds and 10 minutes whereby a mechanical property of said film is modified relative to an unconditioned film.

14. The method of claim 13, wherein said alcohol comprises isopropanol or methanol and said alcohol and said water are present in a ratio of between 4:1 and 1:1.

15. The method of claim 13, further including the step of drying the conditioned film.

16. The method of claim 15, further including the step of soaking the dried conditioned film in a water bath at a temperature of room temperature.

17. The method of claim 13, wherein said film is stretched at least 100% relative to its initial dimensions.

18. A film made in accordance with the method of claim 13.

19. A film made in accordance with the method of claim 1 in combination with a substrate selected from the group consisting of a cosmetic composition; a pharmaceutical or medical composition, drug delivery system, artificial cell, contact lens coating, sustained-release drug delivery system, artificial skin graft; food composition; automotive part; aeronautic component; computer or data storage device, building material, textile, filter material, membrane material, nanomaterial, electronic component, and combinations thereof.
